(19) United States
(12) Patent Application Publication
(10) Pub. No.: US 2022/0221457 A1
(43) Pub. Date: Jul. 14, 2022

(54) MARKER FOR DETECTING PROLIFERATION OF STEM CELL AND HIGH-EFFICIENCY PROLIFERATION METHOD OF STEM CELL USING SAME

(71) Applicant: Samsung Life Public Welfare Foundation, Seoul (KR)

(72) Inventor: Jong Wook CHANG, Seoul (KR)

(73) Assignee: Samsung Life Public Welfare Foundation, Seoul (KR)

(21) Appl. No.: 17/295,122

(22) PCT Filed: Nov. 19, 2019
(86) PCT No.: PCT/KR2019/015859

§ 371 Date: Feb. 17, 2022

(30) Foreign Application Priority Data
Nov. 20, 2018 (KR) 10-2018-0143910

(51) Int. Cl. G01N 33/569

(52) U.S. Cl. CPC G01N 33/56966; G01N 2333/46

(57) ABSTRACT

The present application relates to a marker gene for detecting the proliferative ability of stem cells and uses thereof, and provides a marker detection composition for detecting the proliferative ability of stem cells, a kit for detecting the proliferative ability of stem cells, and a composition for improving the proliferative ability of stem cells, etc. According to the composition or method according to an aspect, stem cells having a high proliferative ability may be easily selected, and the proliferative ability of stern cells may be significantly improved.

### TECHNICAL FIELD

The present disclosure relates to a marker for detecting the proliferative ability of stem cells and a method of highly efficient proliferation of stem cells using the same, This application is based on and claims priority to Korean Patent Application No. 10-2018-0143910 filed on Nov. 20, 2018, and the specification is incorporated by reference herein in its entirety.

### BACKGROUND ART

In order to be used as a cell therapy, research and development on various types of cells are underway, and the cells may be classified into pluripotent stern cells (embryonic stem cells, induced pluripotent stem cells, etc.), multipotent stem cells or adult stem cells (bone marrow/fat-derived stem cells, umbilical cord blood/umbilical cord stem cells, fetal stem cells, etc.), and somatic cells depending on the cell differentiation location, Among these, it is known that human embryonic stem cells have many ethical problems because they are made from embryos that can develop into human organisms, and it will take a long time before pluripotent stem cells are developed as a cell therapy because technology for controlling differentiation thereof has not yet been completed. In addition, although the research and development of cell therapy products using somatic cells has shown many achievements, it is still difficult to put them into practical use due to the low proliferative capability inherent in somatic cells and difficulty in securing raw material tissues. Therefore, as an alternative to overcome these problems, much attention has been focused on the study of adult stem cells.

Among adult stem cells, mesenchymal stem cells have superior proliferative ability compared to somatic cells and are multipotent stem cells capable of differentiating into bone, cartilage, fat, etc., and are much more genetically stabilized than pluripotent stem cells such as embryonic stem cells, and thus have been developed as cell therapeutic agents for regeneration, treatment of myocardial infarction, and treatment of graft versus host disease. Actually, mesenchymal stem cells show higher self-reproduction ability than somatic cells. However, unlike when in the body, when the mesenchymal stem cells are under in vitro culture conditions, the best conditions for maintaining the proliferative properties of cells should be provided, and environmental conditions, such as nutrients, pH, temperature, and osmotic pressure, should be adjusted so as to be close to in vivo conditions, which is quite complicated.

Therefore, various studies are currently underway to improve the in vitro proliferation of mesenchymal stem cells. Korean Patent Registration No. 1138091, inter alia, refers to the types and specific concentrations of some nutrients, such as amino acids, inorganic salts, and vitamins, as a composition in the medium for improving the proliferative ability of undifferentiated mesenchymal stern cells, and Korean Patent Registration No. 1168994 discloses a Cnidium monnieri fruit extract, a Dioscoreae Rhizoma extract, and the like, as components for improving proliferative ability. However, despite these efforts, there is still a demand for a technology capable of selecting or producing mesenchymal stem cells with high proliferative ability,

Under the circumstances, the present inventors have made diligent efforts to develop a technology capable of improving the proliferative ability of stem cells, and as a result, confirmed a significant relationship between the DANCE gene and the proliferative ability of a mesenchymal stem cell, thereby completing the present application.

### DESCRIPTION OF EMBODIMENTS

### Technical Problem

One aspect is to provide a marker detection composition for detecting the proliferative ability of stem cells, comprising a formulation for measuring the expression level of a DANCE gene.

Another aspect is to provide a kit for detecting the proliferative ability of stem cells, comprising a formulation for measuring the expression level of a DANCE gene.

Another aspect is to provide a method for providing information for predicting the proliferative ability of stem cells, comprising measuring the expression level of the DANCE gene of stem cells isolated from an individual.

Another aspect is to provide a composition for improving the proliferative ability of stem cells, comprising a DANCE protein or a gene encoding the protein.

Another aspect is to provide a method for producing stem cells having a high proliferative ability, comprising culturing stem cells in a medium containing the composition.

Another aspect is to provide a method for detecting the proliferative ability of stem cells, comprising administering a formulation for measuring the expression level of a DANCE gene to an individual or stem cells isolated from an individual.

Another aspect is to provide a formulation for measuring the expression level of a DANCE gene for detecting the proliferative ability of stem cells

Another aspect is to provide a method for improving the proliferative ability of stem cells, comprising administering a DANCE protein or a gene encoding the protein to an individual or stem cells isolated from an individual.

Another aspect is to provide the use of a DANCE gene for improving the proliferative ability of stem cells.

Other objects and advantages of the present application will become more apparent by the following detailed description in conjunction with the appended claims and drawings. Contents not described in the present specification will be omitted because they can be sufficiently recognized and inferred by a person skilled in the art of the present application or in a similar technical field.

### Solution to Problem

One aspect provides a marker detection composition for detecting the proliferative ability of stem cells, including a formulation for measuring the expression level of a DANCE (developmental arteries and neural crest epidermal growth factor-like) gene.

As used herein, the term “stem cell” refers to a cell that has the ability to differentiate into two or more different types of cells while having the ability to self-replicate as an undifferentiated cell. The stem cells may be an autologous or allogeneic stem cell, and may be derived from any type of animal, including humans and non-human mammals, and whether the stem cells are derived from an adult or an embryo is not limited thereto. The stem cells may include embryonic stem cells or adult stem cells, and specifically, may be adult stem cells. The adult stem cell may be a mesenchymal stem cell, a human tissue-derived mesenchymal stromal cell, a human tissue-derived mesenchymal stern cell, a multipotent stem cell or an amniotic epithelial cell, and specifically a mesenchymal stern cell, but is not limited thereto. The mesenchymal stern cell may be a mesenchymal stem cell derived from umbilical cord, umbilical cord blood, bone marrow, fat, muscle, nerve, skin, amniotic membrane and placenta, but is not limited thereto.

As used herein, the term “stem cell proliferative ability” specifically refers to the in vitro proliferative ability of stem cells isolated from an individual. Specifically, mesenchymal stern cells, which exist in a small number in adult tissues. such as bone marrow and adipose tissue, have a low proliferation rate in the undifferentiated state, and are difficult to be maintained for a long time in the undifferentiated state, and thus it is difficult to proliferate and culture the same in vitro. Therefore, there is a need to develop a technology capable of selecting stern cells with high proliferative ability.

According to one embodiment, it was found that the expression level of a DANCE gene is closely related to the proliferative ability of stem cells. Specifically, it was elucidated that stem cells having high proliferative ability can be selected through high expression of a DANCE gene in the stem cells. Therefore, the expression level of a DANCE gene can be used to detect the proliferative ability of stem cells.

In the present specification, a DANCE protein is interpreted to include naturally occurring wild-type DANCE and functional variants thereof. In addition, in the present specification, the DANCE gene is interpreted to include a gene encoding a naturally occurring wild-type DANCE protein and a gene encoding a functional variant thereof. Meanwhile, the sequence of the DANCE protein or the gene encoding the same can be obtained from a known database such as GenBank of the National Institutes of Health.

The expression level may include any expression level of the gene encoding the DANCE protein. The expression level may be an expression level at an mRNA or protein phase. Accordingly, the composition may include a formulation for measuring the amount of mRNA of a DANCE gene, the amount of a protein, or a combination thereof. The formulation may be a substance that specifically binds to the transcript of a DANCE gene. For example, the substance may be a primer. a probe, a nucleotide, an antibody or an antigen-binding fragment thereof, a ligand, a receptor, an agonist or an antagonist thereof, or a combination thereof.

The measurement of the mRNA expression level may be a process of determining the presence and expression level of the mRNA of the DANCE gene in stem cells in order to detect the proliferative ability of stem cells, and may include measuring the amount of mRNA. This can be measured by directly separating the mRNA or by using a primer or probe for the mRNA. Methods for analyzing the same may include RT-PCR, competitive RT-PCR, real-time RT-PCR, RNase protection assay (RPA), Northern blotting, a DNA-containing nucleic acid microarray, or a combination thereof. The RT-PCR, which is a method for analyzing RNA, is a method for amplifying and analyzing cDNA obtained by reverse transcription of mRNA by PCR. In the amplification step of the RT-PCR, a primer pair specifically prepared for the gene is used, and by checking the pattern and thickness of a band through electrophoresis after RT-PCR, the mRNA expression and expression level of the gene can be confirmed, and are then compared with those of stem cells having an average proliferative ability, that is, a control group, thereby be easily determining the proliferative ability of stem cells.

As used herein, the term “primer” refers to a nucleic acid sequence having a free 3-terminal hydroxyl group, which is capable of forming a base pair with a template complementary to a specific base sequence, and acts as a starting point for copying the strand of the template. A primer can initiate DNA synthesis in the presence of a reagent for polymerization (Le., DNA polymerase or reverse transcriptase) and four different nucleoside triphosphates at an appropriate buffer and temperature. For example, by using sense and antisense primers having 7 to 50 nucleotide sequences as specific primers for the mRNA of a DANCE gene, PCR amplification can be performed, thereby determining the proliferative ability of stem cells by measuring the amount of desired product generated. The PCR conditions and lengths of the sense and antisense primers can be appropriately selected according to techniques known in the art. The primers may be ones having 10 to 100, 15 to 100, 10 to 80, 10 to 50, 10 to 30, 10 to 20, 15 to 80, 15 to 50, 15 to 30, 15 to 20, 20 to 100, 20 to 80, 20 to 50, or 20 to 30 nt.

As used herein, the term “probe” refers to a fragment of a nucleic acid, such as RNA or DNA, capable of specifically binding to a target nucleic acid, for example, mRNA, and may be labeled so as to identify the presence, content, and expression level of a specific mRNA. The probe may be manufactured in the form of an oligonucleotide probe, a single stranded DNA probe, a double stranded DNA probe, or an RNA probe. For example, by performing hybridization using a probe having a nucleic acid sequence complementary to the mRNA of a DANCE gene, and the expression level of the mRNA may be measured through the degree of hybridization, thereby confirming the proliferative ability. Selection of an appropriate probe and conditions for hybridization can be appropriately selected according to techniques known in the art. The probe may be one having 10 to 100, 15 to 100, 10 to 80, 10 to 50, 10 to 30, 10 to 20, 15 to 80, 15 to 50, 15 to 30, 15 to 20, 20 to 100, 20 to 80, 20 to 50, or 20 to 30 nt.

The measurement of the protein expression level may be a process for determining the presence and expression level of the protein expressed from the DANCE gene in the stem cells in order to detect the proliferative ability of stem cells. This may be, for example, directly separating a DANCE protein, or identifying the DANCE protein by using an antibody or fragment thereof that specifically binds to the DANCE protein. Methods for analyzing the same may include Western blotting, ELISA (enzyme linked immunosorbent assay), radioimmunoassay (RIA), radioimmunodiffusion, Ouchterlony immune diffusion, rocket immunoelectrophoresis, tissue immunostaining, immunoprecipitation assay, complement fixation assay, FACS, mass spectrometry, magnetic bead-antibody immunoprecipitation, protein chip, or a combination thereof. For example, the ELISA may include: direct ELISA using a labeled antibody that recognizes an antigen attached to a solid support; indirect EL ISA using a labeled antibody that recognizes a capture antibody in a complex of antibodies that recognize an antigen attached to a solid support; direct sandwich ELISA using another labeled antibody that recognizes an antigen in a complex of an antibody and an antigen attached to a solid support; and indirect sandwich ELISA using a labeled secondary antibody that is allowed to react with another antibody that recognizes an antigen in a complex of an antibody and an antigen attached to a solid support that and then recognizes the antibody. In addition, the expression level can be detected by a sandwich ELISA method in which, after attaching the antibody to the solid support and reacting the sample, a labeled antibody that recognizes the antigen of the antigen-antibody complex is attached to develop a color enzymatically, or the secondary labeled antibody is attached to the antibody that recognizes the antigen of the antigen-antibody complex to develop a color enzymatically, and the proliferative ability of mesenchymal stem cells can be confirmed by confirming the degree of the protein-antibody complex formation.

The detection method may be performed by investigating and comparing the expression levels of the protein in stem cells having an average level of proliferative ability, that is, the protein expression level in the control group and the target stem cells, and the mRNA or protein level can be expressed as an absolute (e.g., pg/ml) or relative (e.g., relative intensity of a signal) difference between the proteins.

As used herein, the term “antibody” is a term known in the art and refers to a specific immunoglobulin indicated with respect to an antigenic site. The antibody may specifically bind to a DANCE protein or a fragment thereof. The protein fragment of DANCE can be, for example, an immunogenic fragment. The fragment refers to a protein fragment having one or more epitopes that can be recognized by an antibody against the DANCE protein. The DANCE gene is cloned into an expression vector, a DANCE protein encoded by the gene is obtained, and an antibody can be prepared from the protein obtained according to a conventional method in the art.

The form of the antibody includes a polyclonal antibody, a monoclonal antibody, or a recombinant antibody, and all immunoglobulin antibodies are included. The antibody refers to a complete form having two full-length light chains and two full-length heavy chains. In addition, the antibody includes special antibodies such as humanized antibodies. The polyclonal antibody can be prepared by injecting a biomarker protein or fragment thereof, which is an immunogen, into an external host according to conventional methods known to those skilled in the art. As the external host, mammals such as mice, rats, sheep, and rabbits can be used. When injected by intramuscular, intraperitoneal or subcutaneous injection method, the immunogen can be administered with an adjuvant to increase antigenicity in general. Thereafter, blood may be regularly collected from an external host to collect serum showing improved titer and antigen specificity, or antibodies may be isolated and purified therefrom.

The monoclonal antibody can be prepared by techniques for generating immortalized cell lines by fusion known to those skilled in the art. A method for producing a monoclonal antibody will now be briefly described. After purifying the protein, an appropriate amount of about 10 μg is immunized to Balb/C mice, or a polypeptide fragment of the protein is synthesized and bound to bovine serum albumin to immunize the mouse. Then, antigen-producing lymphocytes isolated from the mouse are Immortalized hybridomas are produced by fusion with human or mouse myeloma, and only hybridoma cells that produce the desired monoclonal antibodies are selected and proliferated using ELISA, followed by isolating and purifying monoclonal antibodies from culture. In addition, the monoclonal antibody can be used by obtaining commercially available antibodies against a DANCE protein.

Another aspect provides a kit for detecting the proliferative ability of stem cells, comprising a formulation for measuring the expression level of a DANCE gene.

The kit may be, for example, a microarray capable of measuring the mRNA expression level of a DANCE protein or a gene encoding the DANCE protein. The microarray can be easily manufactured by a person skilled in the art according to a method known in the art by using the DANCE gene. The microarray may include mRNA of a gene encoding a DANCE protein or cDNA of a sequence corresponding to a fragment thereof attached to a substrate as a probe.

For example, when the kit is for measuring the expression level of the mRNA of the DANCE gene, it may include essential elements necessary for performing RT-PCR. The RT-PCR kit may include, in addition to each primer specific for the mRNA of the marker gene, test tubes or other suitable containers, reaction buffers, deoxyribonucleotides (dNTPs), enzymes such as Taq-polymerase and reverse transcriptase, DNase, RNase inhibitors, DEPC-water, or sterile water. In addition, the RT-PCR kit may include a primer pair specific to a gene used as a quantitative control.

In addition, the kit may include an antibody that specifically binds to the DANCE protein, a substrate for immunological detection of the antibody, a suitable buffer, a secondary antibody labeled with a chromogenic enzyme or a fluorescent substance, or a chromogenic substrate. As the substrate, a nitrocellulose membrane, a 96-well plate synthesized from polyvinyl resin, a 96-well plate synthesized from polystyrene resin, or a glass slide glass may be used. As the chromogenic enzyme, peroxidase or alkaline phosphatase may be used. As the fluorescent material. FITC or RITC may be used. As the chromogenic substrate. ABTS (2,2′-azino-bis(3-ethylbenzothiazoline-6-sulfonic acid)), OPD (o-phenylenediamine), or TMB (tetramethyl benzidine) may be used.

It is understood that any of the terms or elements mentioned with regard to the kit, such as those mentioned with regard to the composition, are the same as those mentioned with regard to the composition.

Another aspect provides a method for providing information for predicting the proliferative ability of stem cells, the method comprising the steps of: contacting stem cells isolated from an individual with a DANCE protein or a substance that specifically binds to the mRNA encoding the protein to form a complex thereof; measuring the level of the complex to measure the expression level of the DANCE gene; and comparing the measured expression level with that of the DANCE gene measured in the control group.

The method may also include the step of measuring the expression level of the DANCE gene in a sample by measuring the expression level of the complex.

The measuring of the level of the complex may be performed by detecting a signal from a detectable label attached to the protein or a substance that specifically binds to the mRNA encoding the protein. The measuring of the level of the complex may include measuring the level of the protein or mRNA encoding the protein by separating the complex again, measuring the level of a substance that specifically binds to the protein or mRNA encoding the same, or measuring the level of the complex without separating the same. The measuring may be performed by RT-PCR, competitive RT-PCR, real-time RT-PCR, RNase protection assay (RPA), Northern blotting, Nucleic acid microarray including DNA, Western blotting, enzyme linked immunosorbent assay (ELISA), radioimmunoassay (RIA), radioimmunodiffusion, Ouchterlony immune diffusion, rocket immunity Electrophoresis, tissue immunostaining, Immunoprecipitation Assay, Complement Fixation Assay, FACS, mass spectrometry, magnetic bead-antibody immunoprecipitation, protein chip, or a combination thereof.

The method includes comparing the measured expression level of the DANCE gene with the expression level of the same gene measured in a control group.

The method may include determining that the stem cells have a high proliferative ability when the expression level of the gene measured from the stem cells is higher than that of the same gene measured in the control group. In addition, when the expression level of the gene measured from the stem cell is equal to or lower than that of the same gene measured in the control group, method may include determining that the stem cell has a low proliferative ability, Meanwhile, the control group may refer to stem cells having an average proliferative ability.

The change in the expression level may include a similar stem cell expression level to that of the control group or an increase in the stem cell expression level by 1%, 2%, 3%, 4%, 5%, 10%, 20%, 30%, 40%, 50%, 60%, 70%, 80%, 90%, 100%, 200%, 300%, 400%, 500%, 600%, 700%, 800%, 900%, and 1000% or more, or a similar stem cell expression level to that of the control group or a decrease in the stem cell expression level by 1%, 2%, 3%, 4%, 5%, 10%, 20%, 30%, 40%, 50%, 60%, 70%, 80%, 90%, and 100% or more.

Among the terms or elements mentioned with regard to the information providing method, any of the same as those mentioned with regard to the composition or kit is understood to be the same as those mentioned above with regard to the composition or kit.

Another aspect provides a method for improving the proliferative ability of stem cells, the method comprising the step of culturing stem cells in a composition for improving the proliferative ability of stem cells, the composition comprising a DANCE protein or a gene encoding the protein, or a medium containing the composition for improving the proliferative ability of stem cells.

According to one embodiment, it was found that the proliferative ability of stem cells having low proliferative ability can be restored through control of the expression level of the DANCE gene or treatment with the DANCE protein. Therefore, the DANCE protein or the gene encoding the protein can be used to improve the proliferative ability of stem cells.

The composition may be provided in the form of a medium composition containing a DANCE protein in order to improve the proliferative ability of stem cells.

The medium composition may include a DANCE protein at a concentration ranging from, for example, 1 to 50 ng/ml. The concentration of the DANCE protein may be, for example, 5 to 50 ng/ml, 10 to 45 ng/ml, 15 to 40 ng/ml, 20 to 35 ng/ml, 25 to 30 ng/ml, and specifically, 5 to 15 ng/ml.

The medium composition may include, as a medium containing components capable of proliferating stem cells, but not limited to, a basic medium selected from DMEM (Dulbecco's Modified Eagle's Medium), 80% knockout DMEM, MEM (Minimal Essential Medium), BME (Basal Medium Eagle) , RPMI 1640, F-10, F-12, DMEM-F12, α-MEM (α-Minimal Essential Medium), G-MEM (Glasgow's Minimal Essential Medium), IMDM (Iscove's Modified Dulbecco's Medium), MacCoy's 5A medium, AmnioMax, AminoMaxII complete Medium, Chang's Medium, and MesemCult-XF Medium. In addition, the medium may additionally include 20% knockout serum replacer (Gibco), glutamine, mercaptoethanol, non-essential amino acids, basic fibroblast growth factor (bFGF), and the like.

In addition, the medium may be provided in the form of being included in a viral or non-viral vector in order to transport or deliver the DANCE protein or the gene encoding the protein to stem cells in the body or outside the body. The viral vector may include, for example, adenovirus, vaccinia virus, lentivirus, retrovirus, or herpes simplex virus, and plasmid vector, bacteriophage vector, liposome, bacterial artificial chromosome, yeast artificial chromosomes, etc., may be employed as the non-viral vector.

Among the terms or elements mentioned with regard to the composition for improving the proliferative ability of stem cells and the method for improving the proliferative ability of stem cells, any of the same as those mentioned above is understood to be the same as those mentioned above.

Another aspect provides a method for producing stern cells having a high proliferative ability, the method comprising the step of culturing stern cells in a medium containing a composition for improving the proliferative ability of stem cells, and stem cells produced by the method.

The stem cells may have a high expression level of the DANCE gene, compared to a control group, for example, untreated stem cells, and the high expression level of the DANCE gene may include an increase by 1%, 2%, 3%, 4%, 5%, 10%, 20%, 30%, 40%, 50%, 60%, 70%, 80%, 90%, 100%, 200%, 300%, 400%, 500%, 600%, 700%, 800%, 900%, and 1000% or more, compared to the control.

In addition, the stem cells may have a doubling time of 70 hours or less, 65 hours or less, 60 hours or less, and 55 hours or less, and the stern cells may have a doubling time of, for example, 70 to 40 hours, 70 to 45 hours, 70 to 50 hours, 70 to 55 hours, 70 to 60 hours, 70 to 65 hours, 65 to 40 hours, 65 to 45 hours, 65 to 50 hours, 65 to 55 hours, 65 to 60 hours, 60 to 40 hours, 60 to 45 hours, 60 to 50 hours, 60 to 55 hours, 55 to 40 hours, 55 to 45 hours, 55 to 50 hours, 50 to 40 hours, or 50 to 45 hours.

### Advantageous Effects of Disclosure

According to the composition or method according to an aspect, stern cells having a high proliferative ability can be easily selected, and the proliferative ability of stem cells can be significantly improved. Accordingly, the composition or method according to an aspect can solve a donor variation, which has been pointed out as a problem of the conventional stem cell therapeutic agent, and can improve the production efficiency of stem cell therapeutic agents.

### BRIEF DESCRIPTION OF DRAWINGS

FIG. 1 shows results of observation of the effect of DANCE expression on the adhesion ability of stern cells, as observed by a phase contrast microscope.

FIGS. 2A and 2B show results of confirming the effect of DANCE expression on the proliferative ability of stem cells, specifically FIG. 2A shows the result of the CCK-8 assay and FIG. 2B shows the result of quantifying and evaluating ATP levels in mesenchymal stem cells.

FIGS. 3A and 3B show results of confirming the effect of DANCE expression on apoptosis of stem cells, as confirmed by FITC Annexin V/7-AAD kit, specifically FIG. 3A shows a result of flow cytometry, and FIG. 3B shows a result of quantifying and comparing the result obtained by flow cytometry.

FIGS. 4A and 46 show results of comparison of the expression levels of DANCE of a total of four stern cell groups, specifically FIG. 4A shows a result of confirming the expression level of DANCE by Western blot, and FIG. 4B shows a result of quantifying and comparing the result obtained by Western blot.

FIGS. 5A and 5B show comparison results of the proliferative ability of a total of four stem cell groups, specifically FIG. 5A shows a result of comparing the number of mesenchymal stem cells when harvested after seeding the same amount of cells, and FIG. 5B shows a result of comparing the doubling time of mesenchymal stem cells based on the result shown in FIG. 5A.

FIG. 6 shows a result of observation of the treatment effect on improvement of the adhesion ability of stern cells before or during culture of DANCE, as observed by a phase contrast microscope.

FIGS. 7A and 7B show results of confirmation of the effect of treatment on improvement of the proliferative ability of stem cells before or during culture of DANCE, specifically FIG. 7A shows a result of comparing the number of mesenchymal stem cells, and FIG. 7B shows a result of comparing the doubling times of mesenchymal stem cells.

### BEST MODE

Hereinafter, the present invention will be described in more detail through examples. However, these examples are for illustrative purposes only, and the scope of the present invention is not limited to these examples.

### EXAMPLE 1

### Changes in Adhesion and Proliferation Abilities of Stem Cells according to Expression of DANCE

In this example, it was attempted to confirm the effect of DANCE expression on the adhesion and proliferation abilities of stem cells. Specifically, the mesenchymal stem cells were treated with siRNA targeting DANCE (hereinafter referred to as siDANCE) to suppress the DANCE expression in mesenchymal stem cells, and then changes in the adhesion and proliferation abilities of the mesenchymal stem cells were observed. In addition, when the siDANCE-treated mesenchymal stem cells were again treated by adding DANCE 10 ng/ml to the culture medium (siDANCE+DANCE), changes in the adhesion and proliferation abilities of the mesenchymal stern cells were observed. Specifically, the adhesion ability of mesenchymal stem cells was evaluated using images taken with a phase contrast microscope, and the proliferative ability of mesenchymal stem cells was evaluated by a cell counting kit-8 (CCK-8) and measurement of intracellular ATP levels. Here, the DANCE protein, and the like, were obtained from bio-techne (USA) (Catalog Number: 9006-FB) and used. Meanwhile, in this example, a group (Serum(−)) in which mesenchymal stern cells were cultured in a serum-free medium and a group (siCTRL) treated with scrambled siRNA were set as a control group and a comparison group, respectively.

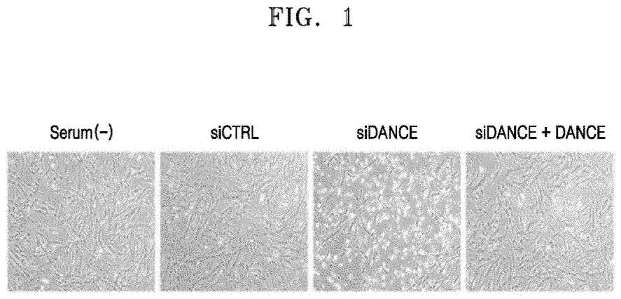

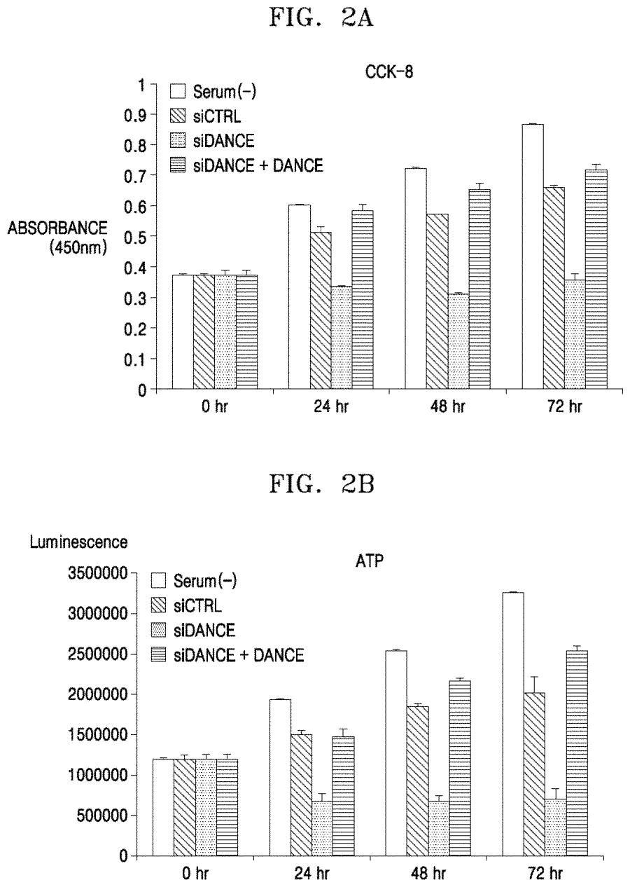

FIG. 1 shows an observation result of the effect of DANCE expression on the adhesion ability of stem cells, as observed by a phase contrast microscope, and FIGS. 2A and 2B show confirmation results of the effect of DANCE expression on the proliferative ability of stem cells. As a result, the adhesion ability of mesenchymal stem cells was decreased by inhibition of the expression of the DANCE gene, and the mesenchymal stern cells with decreased adhesion ability were restored to a similar level as before by treatment with DANCE. In addition, the proliferative ability of mesenchymal stern cells was also lowered by inhibition of the expression of the DANCE gene, similarly to the above, and this change was recovered by the treatment with DANCE.

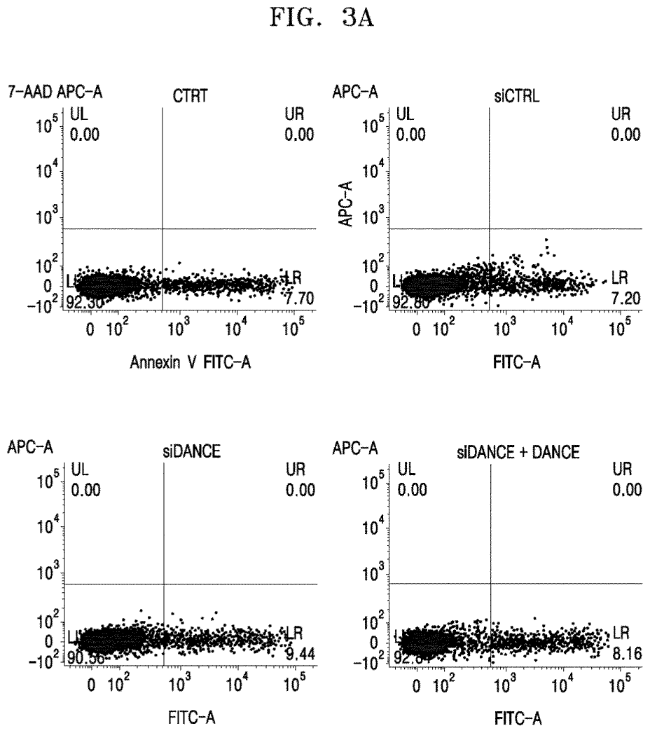

Meanwhile, FIGS. 3A and 3B show confirmation results of the effect of DANCE expression on apoptosis of stem cells, as confirmed by using an FITC Annexin V/7-AAD kit. As a result, the relationship between the apoptosis of mesenchymal stem cells and the expression of the DANCE gene could not be confirmed. These experimental results indicate that the changes in the adhesion and proliferation abilities of the mesenchymal stem cells identified in this example are not the results caused by apoptosis but the results caused by changes in the intrinsic characteristics of cells.

### EXAMPLE 2

### Change in Doubling Time of Stem Cells According to Expression of DANCE

In this example, it was attempted to confirm the effect of the DANCE expression on the doubling time of stem cells. Specifically, the obtained mesenchymal stem cells were randomly classified into four mesenchymal stem cell groups (MSC A, MSC B, MSC C, and MSC D), and then the expression levels of DANCE in the respective classified mesenchymal stem cell groups were measured by Western blot. In addition, it was also attempted to verify again the relationship between the DANCE expression and the proliferative ability of the mesenchymal stern cells by calculating the number of cells harvested after seeding the same amount of cells in the mesenchymal stem cell group, and the doubling time.

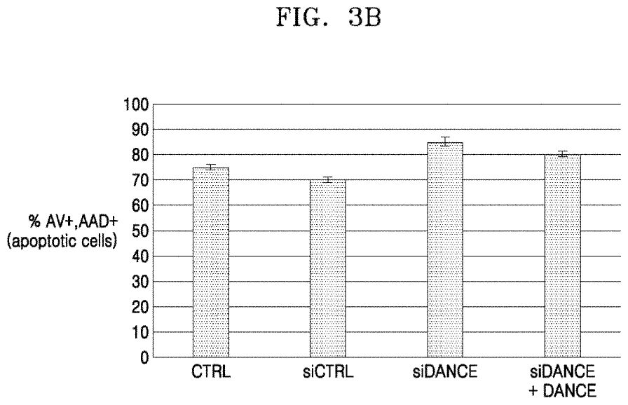

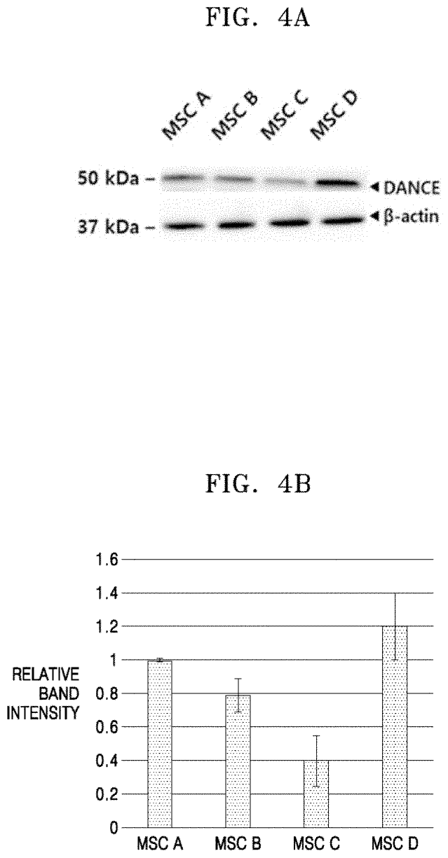

FIGS. 4A and 4B show comparison results of the expression levels of DANCE of a total of four stem cell groups, in which DANCE expression levels were observed to be high in the order of MSC D, MSC A, MSC B, and MSC C. In addition, FIGS. 5A and 5B show comparison results of the proliferative ability of a total of four stem cell groups. As a result, the number of mesenchymal stem cells showed a similar tendency to the expression level of DANCE, and the doubling time of the mesenchymal stern cells showed a tendency opposite to the expression level of DANCE. These experimental results indicate that the proliferative ability of mesenchymal stem cells is closely related to the DANCE expression.

### EXAMPLE 3

### Improvement of Adhesion or Proliferative Ability of Stem Cells According to Treatment with DANCE

In this example, it was attempted to confirm the effect of DANCE treatment on improvement of the adhesion or proliferative ability of stem cells. Specifically, with respect to mesenchymal stem cells with low proliferative ability, etc., changes in their adhesion or proliferative ability were confirmed when 10 ng/ml of DANCE protein was added to the culture medium and treated 1 hour before seeding (Pretreat), or when DANCE proteins were added to on the culture medium during the cultivation process (Treat). The adhesion or proliferative ability of mesenchymal stem cells was evaluated in the same manner as in Example 1 or Example 2, respectively. Meanwhile, in this example, the DANCE protein untreated group (CTRL) was set as a control.

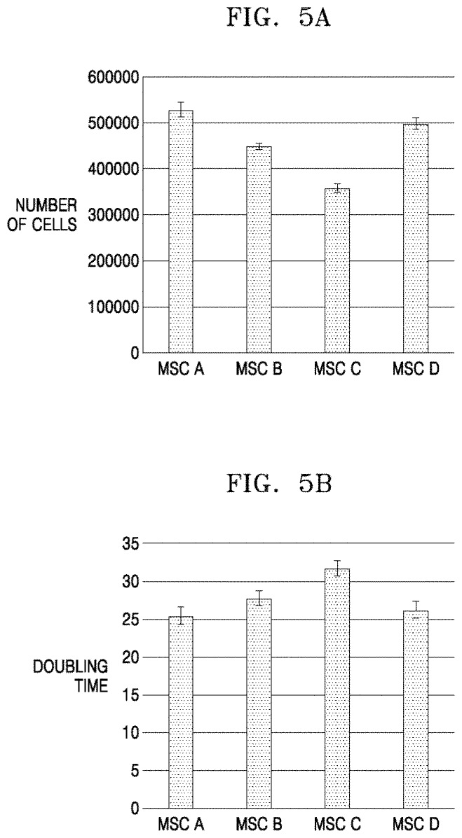

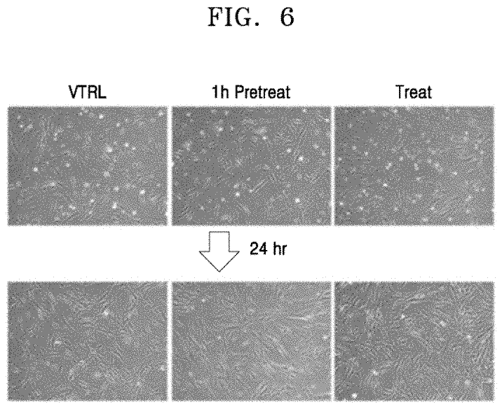

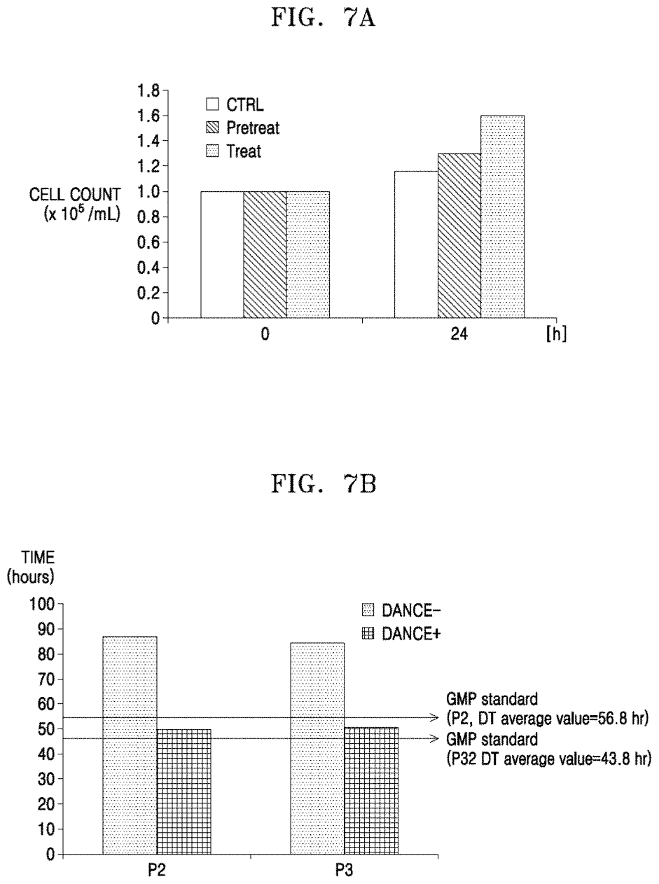

FIG. 6 shows an observation result of the effect of treatment on improvement of the adhesion ability of stem cells before or during culture of DANCE, as observed by a phase contrast microscope, and FIGS. 7A and 7B show confirmation results of the effect of treatment on improvement of the proliferative ability of stem cells before or during culture of DANCE. As a result, the adhesion ability of mesenchymal stem cells was significantly improved in all groups treated with the DANCE protein. In addition, the proliferative ability of mesenchymal stem cells was significantly improved by the pretreatment of the DANCE protein or treatment in the culture process, specifically, the doubling time in P2 was significantly shortened from 87 hours of the prior art to 49 hours and the doubling time in P3 from 84 hours of the prior art to 50 hours. These experimental results indicate that treatment with the DANCE protein before or during the cultivation of mesenchymal stem cells can improve the adhesion or proliferative ability of mesenchymal stem cells.

The above-described present invention has been provided for illustrative purposes only, and a person skilled in the art to which the present invention pertains will be able to understand that other specific forms can be easily modified without changing the technical spirit or essential features of the present invention. Therefore, it should be understood that the above-described embodiments are illustrative and non-limiting in all respects.

1. A marker detection composition for detecting the proliferative ability of stem cells, comprising a formulation for measuring the expression level of a developmental arteries and neural crest epidermal growth factor-like (DANCE) gene.

2. The marker detection composition of claim 1, wherein the expression level is a level of a DANCE protein or an mRNA encoding the protein.

3. The marker detection composition of claim 1, wherein the formulation comprises a substance that specifically binds to a DANCE protein or an mRNA encoding the protein.

4. The marker detection composition of claim 3, wherein the substance is a primer, a probe, a nucleotide, an antibody or an antigen-binding fragment thereof, a ligand, a receptor, an agonist or an antagonist, or a combination thereof, each of which specifically binds to a DANCE protein or an mRNA encoding the protein.

5. The marker detection composition of claim 1, wherein the stem cells are embryonic stem cells or adult stem cells.

6. The marker detection composition of claim 5, wherein the adult stem cells are mesenchymal stem cells derived from a tissue selected from umbilical cord, cord blood, bone marrow, fat, muscle, nerve, skin, amniotic membrane, and placenta.

7. A kit for detecting the proliferative ability of stem cells, the kit comprising the marker detection composition of any one of claims 1 to 6.

8. A method of providing information to predict the proliferative ability of stem cells, comprising:
contacting stern cells isolated from an individual with a developmental arteries and neural crest epidermal growth factor-like (DANCE) protein or a substance that specifically binds to mRNA encoding the protein, to form a complex thereof;
measuring a level of the complex to measure an expression level of the DANCE gene; and
comparing the measured expression level with that of the DANCE gene of a control group.

9. The method of claim 8, further comprising determining that the stem cells have high proliferative ability when the expression level of the DANCE gene measured from the stern cells is higher than that of the control group.

10. The method of claim 8, wherein the measuring of the expression level is performed by RT-PCR, competitive RT-PCR, real-time RT-PCR, RNase protection assay (RPA), Northern blotting, nucleic acid microarray including DNA, Western blotting, enzyme linked immunosorbent assay (ELISA), radioimmunoassay (RIA), radioimmunodiffusion, ouchterlony immune diffusion, rocket immunity electrophoresis, tissue immunostaining, Immunoprecipitation assay, complement fixation assay, FACS, mass spectrometry, magnetic bead-antibody immunoprecipitation, protein chip, or a combination thereof.

11. A composition for improving the proliferative ability of stem cells, the composition comprising Developmental arteries and neural crest epidermal growth factor-like (DANCE) protein or a gene encoding the protein

12. The composition of claim 11, wherein the composition is provided in the form of a medium composition containing a DANCE protein.

13. The composition of claim 11, wherein the stem cells are embryonic stem cells or adult stem cells.

14. The composition of claim 13, wherein the adult stern cells are mesenchymal stem cells derived from a tissue selected from umbilical cord, umbilical cord blood, bone marrow, fat, muscle, nerve, skin, amniotic membrane, and placenta.

15. A method of producing stem cells having a high proliferative ability, the method comprising culturing stem cells in a medium containing the composition of claim 11.
